(19) United States
(12) Patent Application Publication
(10) Pub. No.: US 2026/0345461 A1
(43) Pub. Date: Nov. 5, 2026

(54) COMPOUND CONTAINING ASCORBIC ACID DERIVATIVE

(71) Applicant: DR'S CHOICE CO., LTD., Tokyo (JP)

(72) Inventors: Akio YAMAMOTO, Chiyoda-ku (JP); Masaru NAKAMURA, Chiyoda-ku (JP); Masahiko NAKANO, Chiyoda-ku (JP); Takashi MIYAHARA, Chiyoda-ku (JP)

(73) Assignee: DR'S CHOICE CO., LTD., Tokyo (JP)

(21) Appl. No.: 19/125,494

(22) PCT Filed: Oct. 31, 2022
(86) PCT No.: PCT/JP2022/040630

§ 371 Date: Apr. 29, 2025

(51) Int. Cl. A61K 8/67; A23L 29/00; A61K 31/375

(52) U.S. Cl. CPC A61K 8/676; A23L 29/035; A61K 31/375

(57) ABSTRACT

A food composition, a cosmetic composition, or a pharmaceutical composition containing ascorbic acid 2-glucoside basic amino acid salt and ascorbic acid 2-glucoside alkali metal salt. The composition is stable and has good feel when applied to skin.

### TECHNICAL FIELD

The present invention relates to a stable composition containing ascorbic acid 2-glucosides.

### BACKGROUND ART

Ascorbic acid acts as a reducing agent, a collagen synthesis promoter, and an antioxidant. In particular, the excellent antioxidant effect of ascorbic acid is known as a component that suppresses melanin production in the skin and prevents pigmentation such as spots and freckles, and ascorbic acid is widely incorporated into medicines, foods, cosmetics, and the like.

However, ascorbic acid is unstable in a moisture absorbing state or under alkaline conditions in bulk powder, and an aqueous solution of ascorbic acid is not only stimulated by strong acidity, but also easily oxidized, and causes a Maillard reaction by typical amines involved in decomposition to cause browning, and the browning reaction proceeds. As a result, a cosmetic composition containing ascorbic acid and a product containing moisture are likely to generate an odor caused by browning, and thus the value of the product is lost, and the amount added to the product is limited, and the effect is inevitably affected (Non Patent Literature 1).

From such a viewpoint, a technique of blending a flavonoid glycoside with ascorbic acid in order to suppress browning of ascorbic acid has been reported (Patent Literature 1 and 2). It has also been reported that ascorbic acid 2-glucoside is used instead of ascorbic acid.

However, although aqueous solutions of ascorbic acid 2-glucoside have a lower pH and slightly improved stability, the drawback of browning still remains. Therefore, a technique of blending ascorbic acid 2-glucoside with a strong base substance, lecithin, an ether compound, an ester compound, citric acid, or the like has also been reported (Patent Literature 3 to 6).

### CITATION LIST

### Patent Literature

[Patent Literature 1] JP 04-099771 A
[Patent Literature 2] JP 04-099730 A
[Patent Literature 3] JP 2001-199863 A
[Patent Literature 4] JP 2002-265344 A
[Patent Literature 5] JP 2005-220029 A
[Patent Literature 6] JP 2012-246248 A

### Non Patent Literature

[Non Patent Literature 1] Journal for the Integrated Study of Dietary Habits, Vol. 26, No. 1, 7-10 (2015)

### SUMMARY OF INVENTION

### Technical Problem

However, the browning reaction of ascorbic acid 2-glucoside could not be sufficiently suppressed by any means, and the browning could not be sufficiently suppressed while maintaining the excellent action and effect of a composition such as a cosmetic composition containing the ascorbic acid 2-glucoside.

Therefore, the present inventor produced and isolated a basic amino acid salt of ascorbic acid 2-glucoside, and found that this substance is not browned and is stable as compared with conventional ascorbic acid and ascorbic acid 2-glucoside. However, there is still room for improvement in the stability, and there is also room for improvement in the feel during use when applied to the skin.

Therefore, an object of the present invention is to provide a stable composition containing ascorbic acid 2-glucosides with a pleasant feel during use.

### Solution to Problem

Therefore, the present inventors conducted studies on the stability of ascorbic acid 2-glucoside basic amino acid salt and the feel during use when the ascorbic acid 2-glucoside basic amino acid salt was blended with various components and applied to the skin, and found that when the ascorbic acid 2-glucoside basic amino acid salt and the ascorbic acid 2-glucoside alkali metal salt are used in combination, the stability is improved and the feel during use when the ascorbic acid 2-glucoside basic amino acid salt and the ascorbic acid 2-glucoside alkali metal salt are applied to the skin is also improved, thereby completing the present invention.

The present invention provides the following inventions [1] to [6].
[1] A food composition, a cosmetic composition, or a pharmaceutical composition containing ascorbic acid 2-glucoside basic amino acid salt and ascorbic acid 2-glucoside alkali metal salt.
[2] The composition according to [1], in which the ascorbic acid 2-glucoside basic amino acid salt is one or more selected from ascorbic acid 2-glucoside lysine salt, ascorbic acid 2-glucoside arginine salt, ascorbic acid 2-glucoside histidine salt, and ascorbic acid 2-glucoside tryptophan salt.
[3] The composition according to [1] or [2], in which the ascorbic acid 2-glucoside alkali metal salt is one or two selected from ascorbic acid 2-glucoside sodium salt and ascorbic acid 2-glucoside potassium salt.
[4] The composition according to any one of [1] to [3], in which pH of the composition is 5.0 or more and 7.5 or less.
[5] The composition according to any one of [1] to [4], in which the ascorbic acid 2-glucoside basic amino acid salt is ascorbic acid 2-glucoside arginine salt.
[6] The composition according to any one of [1] to [5], in which the ascorbic acid 2-glucoside alkali metal salt is ascorbic acid 2-glucoside sodium salt.

### Advantageous Effects of Invention

Since the aqueous solution containing ascorbic acid 2-glucoside basic amino acid salt and ascorbic acid 2-glucoside alkali metal salt remarkably suppresses coloring and has a weak acidity to a neutral pH, the aqueous solution has various functions of ascorbic acids, such as a whitening effect through melanin production suppression and a wrinkle improving effect through collagen production activation, and is useful as a blending component of safe food, medicine, or cosmetic without skin irritation.

### BRIEF DESCRIPTION OF DRAWINGS

FIG. 1 is a diagram showing an FT/IR spectrum of ascorbic acid 2-glucoside arginine salt powder.

### DETAILED DESCRIPTION OF THE INVENTION

One aspect of the present invention is a composition containing ascorbic acid 2-glucoside basic amino acid salt and ascorbic acid 2-glucoside alkali metal salt. This composition is particularly useful as a food composition, a cosmetic composition, or a pharmaceutical composition.

Ascorbic acid in the present invention is a substance that is a compound having an IUPAC name of (R)-3, 4-dihydroxy-5-((S)-1,2-dihydroxyethyl) furan-2 (5H)-one, and is also referred to as vitamin C or L-ascorbic acid. Ascorbic acid acts as a reducing agent, a collagen synthesis promoter, and an antioxidant as described above, and in particular, the excellent antioxidant effect of ascorbic acid is known as a component that suppresses the melanin production in the skin and prevents pigmentation such as spots and freckles, and is widely used for medicines, foods, and cosmetics.

Ascorbic acid 2-glucoside is a substance in which glucose is bonded to the carbon atom at the 2-position of ascorbic acid, and is metabolized in vivo to become ascorbic acid, and thus is regarded as a provitamin of ascorbic acid. The ascorbic acid 2-glucoside basic amino acid salt used in the present invention is a basic amino acid salt of ascorbic acid 2-glucoside.

Here, examples of the ascorbic acid 2-glucoside basic amino acid include one or more selected from ascorbic acid 2-glucoside lysine salt, ascorbic acid 2-glucoside arginine salt, ascorbic acid 2-glucoside histidine salt, and ascorbic acid 2-glucoside tryptophan salt. Among them, ascorbic acid 2-glucoside lysine salt and ascorbic acid 2-glucoside arginine salt are more preferable, and ascorbic acid 2-glucoside arginine salt is still more preferable.

The constituent molar ratio of ascorbic acid 2-glucoside and basic amino acid in the basic amino acid salt of ascorbic acid 2-glucoside is preferably 1:1.5 to 1.5:1, and more preferably 1:1.2 to 1.2:1.

The ascorbic acid 2-glucoside basic amino acid salt can be produced by reacting a basic amino acid with ascorbic acid 2-glucoside. Specifically, ascorbic acid 2-glucoside and a basic amino acid are added at 40 to 60 °C in ion-exchanged water in which the concentration of dissolved oxygen is reduced by ultrasonic treatment or the like, and the pH is adjusted to 5.22 to 5.55, thereby adjusting the amount of the basic amino acid to be added and allowing the reaction to proceed. After completion of the reaction, the ascorbic acid 2-glucoside basic amino acid salt can be isolated as a powder by freeze drying, spray drying, or the like.

The obtained ascorbic acid 2-glucoside basic amino acid salt powder is stable for a long period of time and is not browned, and thus is useful as a raw material for blending in a cosmetic or pharmaceutical composition at a high concentration. In the solution, there was a limit to the upper limit of the concentration in order to mix with other bases. In order to store the raw material in a liquid state, it is necessary to add a preservative or the like, to sterilize the raw material through filtration, to fill a sterile container with the raw material, or to store the raw material at a low temperature, but by powdering the raw material, the raw material can be stably and compactly stored at room temperature without adding other components.

The content of the ascorbic acid 2-glucoside basic amino acid salt in the composition of the present invention is preferably 1 to 40% by mass, more preferably 2 to 35% by mass, and still more preferably 5 to 308 by mass from the viewpoint of stability, feel during use, and the function of the ascorbic acid 2-glucoside basic amino acid salt as ascorbic acid.

Examples of the ascorbic acid 2-glucoside alkali metal salt include ascorbic acid 2-glucoside sodium salt, ascorbic acid 2-glucoside potassium salt, and ascorbic acid 2-glucoside lithium salt. Among them, ascorbic acid 2-glucoside sodium salt and ascorbic acid 2-glucoside potassium salt are preferable, and ascorbic acid 2-glucoside sodium salt is more preferable.

The constituent molar ratio of ascorbic acid 2-glucoside and alkali metal in the ascorbic acid 2-glucoside alkali metal salt is preferably 1:1.5 to 1.5:1, and more preferably 1:1.2 to 1.2:1.

The ascorbic acid 2-glucoside alkali metal salt can be produced by a method described in JP 2002-88095 A, Japanese Patent No. 6895436, or the like. For example, an alkali metal salt such as sodium hydroxide, sodium carbonate, or sodium hydrogen carbonate may be reacted with ascorbic acid 2-glucoside. Specifically, ascorbic acid 2-glucoside and an alkali metal salt are added at 40 to 60 °C in ion-exchanged water with a reduced dissolved oxygen concentration such as sonication, and the pH is adjusted to 6.0 to 6.5, thereby adjusting the amount of the alkali metal salt to be added and allowing the reaction to proceed. After completion of the reaction, the ascorbic acid 2-glucoside alkali metal salt can be isolated as a powder by freeze drying, spray drying, or the like.

Ascorbic acid 2-glucoside alkali metal salt is known to have the same physiological activity as ascorbic acid, but it is not known that the stability is improved and the feel during use is improved by using ascorbic acid 2-glucoside basic amino acid salt in combination.

The content of the ascorbic acid 2-glucoside alkali metal salt in the composition of the present invention is preferably 1 to 50% by mass, more preferably 2 to 45% by mass, and still more preferably 5 to 408 by mass from the viewpoint of stability, feel during use, and the function of the ascorbic acid 2-glucoside alkali metal salt as ascorbic acid.

The molar content ratio (a/b) of the ascorbic acid 2-glucoside basic amino acid salt (a) and the ascorbic acid 2-glucoside alkali metal salt (b) in the composition of the present invention is preferably 0.2 to 5.0, more preferably 0.3 to 3.0, and still more preferably 0.5 to 2.0 from the viewpoint of the discoloration prevention effect of the composition, the effect of exhibiting a function as ascorbic acid, and the feel during use such as sticky feeling.

The pH of the composition of the present invention is preferably 5.0 to 7.5, more preferably 5.0 to 6.5, and still more preferably 5.0 to 6.0 from the viewpoint of a discoloration prevention effect and a reduction of irritation when applied to the skin.

Examples of the composition containing ascorbic acid 2-glucoside basic amino acid salt and ascorbic acid 2-glucoside alkali metal salt include a food composition, a pharmaceutical composition, and a cosmetic composition. Examples of the form of these compositions include liquid compositions, emulsion compositions, powder compositions, and solid compositions.

Examples of the food composition include functional foods and foods for specified health uses. Examples of the pharmaceutical composition include preparations for oral administration such as tablets, granules, and syrups, preparations for transdermal administration such as creams, ointments, and solutions, eye drops, and injections.

Examples of the cosmetic composition include a liquid cosmetic, an emulsion cosmetic, a powdery cosmetic, and a solid cosmetic. In addition to the ascorbic acid 2-glucoside basic amino acid salt and the ascorbic acid 2-glucoside alkali metal salt, these cosmetics preferably contain ascorbic acid or a salt thereof, water, an oil agent, a surfactant, a moisturizing component, alcohols, polyols, an ultraviolet absorber, an ultraviolet protecting agent, various powders, various cosmetic components, an antioxidant, an antibacterial agent, a fragrance, and the like.

In addition, the cosmetic of the present invention is more preferably a skin cosmetic from the viewpoint of sufficiently obtaining the functions of ascorbic acid 2-glucoside basic amino acid salt and ascorbic acid 2-glucoside alkali metal salt. In addition, the form of the cosmetic is preferably an aqueous liquid cosmetic, an oil-in-water emulsion cosmetic, or a water-in-oil emulsion cosmetic.

### EXAMPLES

Next, the present invention will be described in more detail with reference to Examples, but the present invention is not limited to these Examples.

### Production Example 1

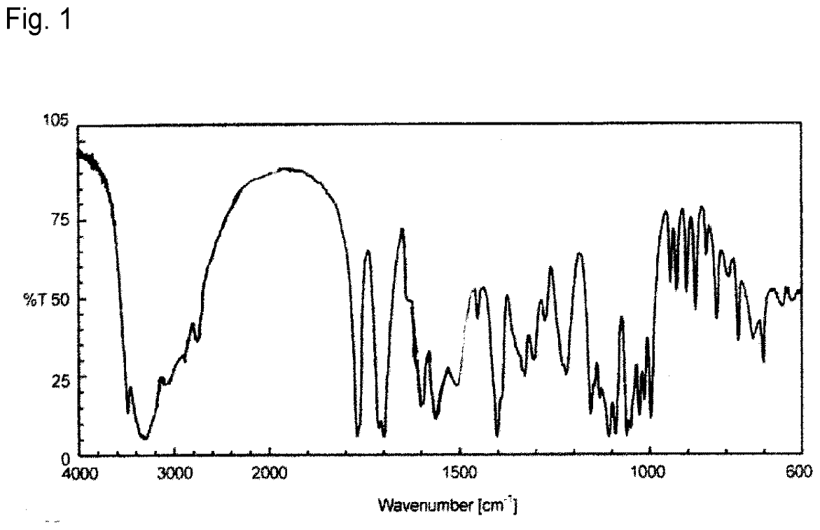

The ion-exchanged water was degassed with an ultrasonic wave having a power of 0.5 kW to 1.0 kW. Although degassing was performed for 20 to 40 minutes at the time of setting 0.8 kW, the degassing was confirmed by measuring with a dissolved oxygen concentration meter, it was confirmed that the dissolved oxygen concentration value was 0.5 ppm or less, and ion-exchanged water was obtained. 50 ml of this water was set to 45 to 60 °C, and more preferably 50 °C±0.5. 5.0 g of ascorbic acid 2-glucoside was collected, stirred and dissolved, and then 14.464 mmol of arginine was added thereto. At this time, the pH is preferably 5.22 to 5.55, and more preferably 5.35. At this time, the molar concentration of arginine to be added is finely adjusted by pH. This solution was freeze-dried to obtain ascorbic acid 2-glucoside arginine salt powder (7.55 9, 90.03%). The FT/IR spectrum of the obtained ascorbic acid 2-glucoside arginine salt powder is shown in FIG. 1. In the spectrum result, 1720 cm-1 (Non-ionic COOH, antisymmetric stretching vibration) and a specific band of 1400 cm⁻¹ were observed, and introduction of an ionic bond of arginine was confirmed.

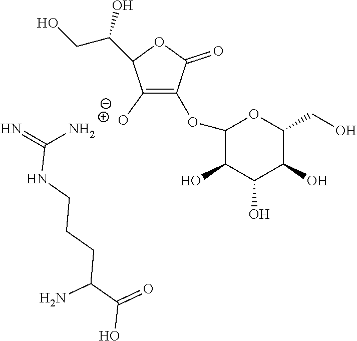 [SMILES: N=C([NH3+])NCCCC(N)C(=O)O ; O=C1OC([C@@H](O)CO)C(O)=C1OC1O[C@H](CO)[C@@H](O)[C@H](O)[C@H]1O]

### Production Example 2

The ion-exchanged water treatment method used for dissolution was performed in the same manner as in Production Example 1. 100 ml of this ion-exchanged water was set to 50 °C±0.5, 5.0 g of ascorbic acid 2-glucoside was added, stirred and dissolved, and then 17.97 mmol of lysine was added. The pH was 5.1 to 5.6, but the molar concentration was finely adjusted to pH5.3. This solution was freeze-dried to obtain a powder of ascorbic acid 2-glucoside lysine salt (7.62 g, 98.42%).

### Example 1

(Mixture of Ascorbic Acid 2-Glucoside Arginine Salt (ASA2-G·Arg) and Ascorbic Acid 2-Glucoside Sodium Salt (ASA2-G·Na))

The solubility of ASA2-G is 879 g/L (25 °C), that is, 46.8% by wt. The solubility of ASA2-G·Arg and ASA2-G·Na in water was improved, and ASA2-G·Arg and ASA2-G·Na could be dissolved in water at a high concentration of 70% by wt or more. In addition, it was possible to dissolve ASA2-G·Arg and ASA2-G·Na in water at the same time, and it was also possible to increase the total concentration of the ascorbic acid 2-glucoside derivatives.

A serum obtained by mixing ASA2-G·Arg and ASA2-G·Na (the total concentration of these components was 40% by wt) was prepared, applied to the faces of six subjects, and questionnaire results of the feel during use (stickiness) were obtained. Five scores from good to bad (the higher the score, the better) were averaged. The weight ratios of the mixtures was four kinds of ASA2-G. Arg:ASA2-G·Na=100:0, 80:20, 60:40, 40:60, 20:80, and 0:100 (test sample 2-5), and were compared with test sample 1 in which ASA2-G·Arg was dissolved alone.

Further, a transparent glass container was filled with test sample 1-5, the test sample was stored at 40 °C for 2 weeks, and the change in coloration was observed with the naked eye.

The obtained results are shown in Table 1.

TABLE 1

| Test sample No. | | 1 | 2 | 3 | 4 | 5 |
|---|---|---|---|---|---|---|
| Weight ratio | ASA2-G•Arg | 100 | 80 | 60 | 40 | 20 |
| of mixture | ASG2-G•Na | 0 | 20 | 40 | 60 | 80 |

| | | | | | |
|---|---|---|---|---|---|
| Average score of feel during use | 3.5 | 4.7 | 4.8 | 4.9 | 4.9 |
| Stability (coloring) | Δ | ◯ | ◯ | ◯ | ◯ |

Δ: Slightly yellow,
◯: Pale yellow,
⊙: Colorless and transparent

Some subjects felt that test sample 1 was sticky after application. In the case of test sample 2-5, the feel during use was almost the same, and there was no subject who felt that the test sample was sticky. It was found that when ASA2-G·Na was blended to ¼ or more of ASA2-G·Arg, the feel during use was significantly improved. Further, in the case of test sample 2-5, the degree of browning when stored at 40 °C for 2 weeks was almost the same, and there was no difference.

### Example 2

A blending example and a production method in the case of blending 5% by mass of ascorbic acid 2-glucoside arginine salt (ASA2-G·Arg) and 5% by mass of ascorbic acid 2-glucoside sodium salt (ASA2-G·Na) to produce a serum are shown below.
(1) Blending (unit: % by mass)
1. Water 67.98%
2. Propanediol 17.0%
3. ASA2-G·Arg 5.0%
4. ASA2-G·Na 5.0%
5. Glycerin 3.9%
6. Na citrate 0. 5%
7. 1,3-BG 0.30%
8. Glycosphingolipids 0.15%
9. Xanthan gum 0.12%
10. Na hyaluronate 0.02%
11. Citric acid 0.02%
12. Acetylhydroxyproline 0.01%
13. Hydrolyzed collagen 0.001%
14. Hydrolyzed elastin 0.00001%

### (2) Production Method

The oil phase components are mixed and heated to approximately 80 °C. Next, the remaining raw materials (water-soluble components) are mixed and heated to approximately 80 °C. While maintaining the temperature at 80 °C, the oil phase component is gradually added to the water-soluble component and emulsified while stirring with a homomixer. Finally, the temperature is cooled to room temperature, then ASA2-G·Arg and ASA2-GNa are added, and the mixture is well mixed, and the mixture is homogenized to obtain a product.

1. A food composition, a cosmetic composition, or a pharmaceutical composition comprising:
ascorbic acid 2-glucoside basic amino acid salt; and
ascorbic acid 2-glucoside alkali metal salt.

2. The composition according to claim 1, wherein the ascorbic acid 2-glucoside basic amino acid salt is one or more-selected from the group consisting of ascorbic acid 2-glucoside lysine salt, ascorbic acid 2-glucoside arginine salt, ascorbic acid 2-glucoside histidine salt, and ascorbic acid 2-glucoside tryptophan salt.

3. The composition according to claim 1, wherein the ascorbic acid 2-glucoside alkali metal salt is one or two selected from the group consisting of ascorbic acid 2-glucoside sodium salt and ascorbic acid 2-glucoside potassium salt.

4. The composition according to claim 1, wherein pH of the composition is 5.0 or more and 7.5 or less.

5. The composition according to claim 1, wherein the ascorbic acid 2-glucoside basic amino acid salt is ascorbic acid 2-glucoside arginine salt.

6. The composition according to claim 1, wherein the ascorbic acid 2-glucoside alkali metal salt is ascorbic acid 2-glucoside sodium salt.
